(19) United States
(12) Patent Application Publication
(10) Pub. No.: US 2008/0241226 A1
(43) Pub. Date: Oct. 2, 2008

(54) Methods and Kits For Administering Probiotics

(76) Inventors: Susan L. Abeln, Loveland, OH (US); Duane Larry Charbonneau, Mason, OH (US); Ker-Sang Chen, West Chester, OH (US); James Patrick Ebel, Lebanon, OH (US); Mary Elaine Freeland, Loveland, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/056,702

(22) Filed: Mar. 27, 2008

Related U.S. Application Data
(60) Provisional application No. 60/920,177, filed on Mar. 27, 2007.

(51) Int. Cl. A61K 9/00; A61K 35/74

(52) U.S. Cl. 424/439; 424/93.4

(57) ABSTRACT

Methods for administering probiotics comprising the steps of: administering a loading dose of a loading probiotic for a loading time period; and administering a dose of a botanical and/or additional materials for the loading time period are disclosed. The methods also include administering a maintenance dose of a maintenance probiotic, and/or a botanical and/or an additional material for a maintenance time period. Also disclosed are kits for use in administering probiotics.

### CROSS REFERENCE TO RELATED APPLICATIONS

This application claims the benefit of U.S. Provisional application No. 60/920,177, filed on Mar. 27, 2007.

### FIELD OF THE INVENTION

The present invention relates generally to methods and kits for administering probiotics. More particularly, the invention relates to methods and kits using a loading dose of probiotic in combination with a botanical and/or additional material. Most particularly, the present invention relates to methods and kits providing a loading and maintenance dose regimen of probiotic in combination with a botanical and/or additional material.

### BACKGROUND OF THE INVENTION

The use of various supplements for preventing and/or alleviating various symptoms associated with particular health problems is generally well known. Problems with the digestive system and gastrointestinal tract can be particularly unpleasant and since ancient times, various foods, herbs, natural compounds and methods have been known to treat the digestive system.

An integral part of mammalian digestive systems is the balance of bacteria therein that are essential to the proper health of the gastrointestinal system and the overall heath of the individual. The intestinal flora is made up of various combinations of bacteria including at least about 400 species of living bacteria, many of which have a symbiotic relationship with the body. Such beneficial bacteria include lactic acid bacteria such as bifido bacteria. These beneficial types of bacteria provide a number of benefits, including enhancing digestion and nutrient absorption, improving bowel function, and supporting natural immunity. Beneficial bacteria can also produce vitamins and assist in the digestion of proteins and sugars. Additionally, and very importantly, beneficial bacteria can inhibit the growth of pathogenic microorganisms including bacteria and other microbes, viruses and protozoa. Beneficial bacteria can inhibit the growth of the pathogenic microorganisms in various ways including secreting substances that reduce the pH of the gastrointestinal tract, and secreting bacteriocins, thereby making the gastrointestinal system less hospitable to pathogenic organisms and/or killing them. Disruption of the balance of commensal bacteria can lead to numerous problems and diseases, ranging from mild to moderate gastrointestinal symptoms to serious infection by pathogenic microorganisms.

Beneficial bacteria, generally referred to as probiotic bacteria or “probiotics”, have been used to improve the overall health of mammals, and in particular have been used because of their beneficial effects on the gastrointestinal system. Gastrointestinal diseases and/or conditions that may be prevented or therapeutically managed using probiotics include abdominal cramps, abdominal discomfort, abdominal distension, antibiotic associated diarrhea (AAD), belching, bloating, celiac disease, cholecystitis, Clostridium difficile associated diarrhea (CDAD), Crohns disease, constipation (including chronic or functional constipation), diarrhea (including chronic or functional diarrhea), disorders of motility, diverticulitis or diverticular disease, duodenal ulcers, dyspepsia (including functional dyspepsia), erosive esophagitis, excess flatus, feeling of incomplete bowel movement, gall bladder disease, gastroesophageal reflux disease (GERD), gastroparesis, gastritis, gastric ulcers, halitosis, heartburn (including frequent heartburn), hypersecretory conditions such as Zollinger-Ellison syndrome, improvement or modulation of gut-barrier function, inflammatory bowel disease (IBD), irritable bowel syndrome (IBS) indigestion, lactose intolerance, mechanical irritation of the bowel, motion sickness, multiple endocrine adenomas, nausea, pain, posterior laryngitis, post-infection colitis, pouchitis, small intestine bacterial overgrowth (SIBO) or small bowel bacterial overgrowth (SBBO), spasm, spastic colon, stomach problems, straining to have a bowel movement, systemic mastocytosis, sweating when having a bowel movement, ulcerative colitis (UC), urgency to have a bowel movement, visceral hypersensitivity, viral diarrhea, vomiting, reaction to therapeutic compositions, and the like.

In addition, the health benefits of probiotic bacteria have been increasingly recognized to include not only benefits for the gastrointestinal system, but also beneficial effects for healthy individuals desiring to improve overall health and wellbeing, and for individuals with a wide range of sub-optimal health conditions. Such sub-optimal heath conditions for which probiotic bacteria can be used as either treatments or preventative therapies include disorders of immunoregulation or immunomodulation in particular as relating to allergies, particularly food allergies, seasonal allergies, environmental allergies, asthma, atopic dermatitis, eczema or other atopic disease or autoimmune disorders, particularly osteoarthritis, rheumatoid arthritis, lupus, multiple sclerosis and fibromyalgia, athralgias or other inflammatory conditions of the muscles or joint, chronic pelvic pain syndrome, depression, stress or altered stress responses such as somatization, autism spectrum disorders, impaired mentation, impaired memory or other disorders of mental wellbeing, attention deficit/hyperactivity disorder, feeling tired and weak, chronic fatigue syndrome, respiratory infection such as common cold, systemic yeast, bacterial or viral infection including candiasis, urinary tract infection, cystitis, vaginosis and vaginitis, obesity, eating disorders such as anorexia and bulimia or other disorders of malnourishment, disorders of the skin such as acne, dandruff, poor hydration, dental caries and oral health, modulation or reduction of risk factors for cardiovascular disease, prevention of osteoporosis and cancer prevention.

However, the use of probiotic bacteria for overall health, symptom therapy, and health maintenance can be problematic. Typically, probiotic bacteria is administered for several weeks before a beneficial effect is noticed by the user. Additionally, a number of users beginning therapy with probiotic bacteria experience undesirable adjustment effects, particularly symptoms including altered sensations related to the passage of bowel movements, feelings of incomplete bowel movement, abdominal cramps, abdominal discomfort, abdominal distension, anxiety, belching, bloating, constipation, depression, diarrhea, disorders of motility, drowsiness, dyspepsia, erosive esophagitis, excess flatus, excessive drainage syndrome, feeling tired and weak, gastritis, gastroesophageal reflux, gastroparesis, headache, heartburn, hypersecretory conditions, indigestion, insomnia, irritability, itching, mechanical irritation of the bowel, nausea, nervousness, pain, rash, sleepiness, spasm, stomach problems, straining to have a bowel movement, sweating when having a bowel movement, urgency to have a bowel movement, vomiting, reactions to therapeutic compositions, and various combinations of these problems. Such effects are often more pronounced in the early stages of use of probiotic bacteria, and for many users, such unpleasant effects result in discontinuation of product use, or delay the time it takes for the user to notice benefits, because the user often feels worse before feeling better. Therefore, dosing of probiotic bacteria must generally be controlled and has traditionally been relatively low initially in order to balance imparting of beneficial effects while minimizing undesirable adjustment effects.

While attempts have been made to reduce the time to achieve a benefit of using probiotic bacteria, many of these efforts have been focused on upon improving probiotic bacteria formulations to protect viable microorganisms from the gastric and intestinal environment, including the stomach, bile salts, and digestive system. However, these approaches do not reduce the time to perception of noticeable benefits, or mitigate the negative adjustment effects.

Thus, there remains a need for an effective means of reducing the time to effectiveness and/or perceived effectiveness of treatment with probiotic bacteria; there remains a need for achieving a higher level of effectiveness; there remains a need to decrease the number of users who experience negative adjustment effects; there remains a need for minimizing the number of days any negative adjustment effects occur; there remains a need for minimizing the amount, type, and severity of negative adjustment effects; and there remains a need for providing additional health benefits that are synergistic to those of probiotic bacteria.

### SUMMARY OF THE INVENTION

The present invention comprises methods of administering a probiotic including the steps of:
a. administering a loading dose of a loading probiotic for a loading time period; and
b. administering a dose of a botanical for the loading time period.

The present invention also comprises methods of administering a probiotic including the steps of:
a. administering a loading dose of a loading probiotic for a loading time period; and
b. administering a dose of an additional material for the loading time period.

The present invention comprises methods of administering a probiotic including the steps of:
a. administering a loading dose of a loading probiotic for a loading time period;
b. administering a dose of a botanical for the loading time period; and
c. administering a dose of an additional material for the loading time period.

The methods of the invention also include administering a maintenance dose of a maintenance probiotic for a maintenance time period; administering a dose of a botanical for a maintenance time period; administering a dose of an additional material for a maintenance time period; and combinations thereof.

The methods of the invention can also include optionally administering a dose of a pre-loading composition for a pre-loading time period.

The methods of the invention improve tolerability and perception of benefits of administering probiotics.

The invention also includes kits for use in administering probiotics; including loading doses of a loading probiotic to be administered for a loading time period; doses of a botanical to be administered for a loading time period; doses of an additional material to be administered for a loading time period; a compliance aid; instructions for use of the kit and the components thereof; and combinations thereof.

The kits can also include doses of a pre-loading composition to be administered for a pre-loading time period before the loading time period; doses of a probiotic to be administered for a maintenance time period; doses of a botanical to be administered for a maintenance time period; doses of an additional material to be administered for a maintenance time period; a compliance aid; instructions for use of the kit and the components thereof; and combinations thereof.

### BRIEF DESCRIPTION OF THE DRAWINGS

FIG. 1 is a top plan view of an embodiment of a compliance aid of the present invention.

FIG. 2 is a right side elevational view thereof.

FIG. 3 is a front side elevational view thereof.

FIG. 4 is a top plan view of another embodiment of a compliance aid of the present invention.

FIG. 5 is a right side elevational view thereof.

FIG. 6 is a front side elevational view thereof.

FIG. 7 is a top plan view of another embodiment of a compliance aid of the present invention.

FIG. 8 is a right side elevational view thereof.

FIG. 9 is a front side elevational view thereof.

FIG. 10 is a top plan view of another embodiment of a compliance aid of the present invention.

FIG. 11 is a right side elevational view thereof.

FIG. 12 is a front side elevational view thereof.

FIG. 13 is a top plan view of another embodiment of a compliance aid of the present invention.

FIG. 14 is a right side elevational view thereof.

FIG. 15 is a front side elevational view thereof.

FIG. 16 is a top plan view of another embodiment of a compliance aid of the present invention.

FIG. 17 is a right side elevational view thereof.

FIG. 18 is a front side elevational view thereof.

FIG. 19 is a top plan view of another embodiment of a compliance aid of the present invention.

FIG. 20 is a right side elevational view thereof.

FIG. 21 is a front side elevational view thereof.

FIG. 22 is a top plan view of another embodiment of a compliance aid of the present invention.

FIG. 23 is a right side elevational view thereof.

FIG. 24 is a front side elevational view thereof.

### DETAILED DESCRIPTION OF THE INVENTION

The methods of the present invention comprise administering a loading dose of a loading probiotic; a dose of botanical, optionally a dose of an additional material selected from the group consisting of vitamins, minerals, metals, elements, essential fatty acids, essential amino acids, sensates, prebiotics, carotenoids, and combinations thereof, and optionally an additional material for a loading time period. The methods also include administering a maintenance dose of a maintenance probiotic for a maintenance time period. The methods also include optional administration of a botanical; and optional administration of one or more additional materials selected from the group consisting of vitamins, minerals, metals, elements, essential fatty acids, essential amino acids, sensates, prebiotics, carotenoids, and combinations thereof; for a maintenance time period. The methods also include optional administration of a pre-loading composition for a pre-loading time period.

“Administering” as used herein means any method which delivers the compositions of the present invention to the user in such a manner so as to be effective in preventing and/or alleviating gastrointestinal distress and associated symptoms; preventing and/or alleviating negative adjustment effects associated with administration of probiotics; providing and/or enhancing perceived effectiveness of the compositions; achieving a higher level of effectiveness of the compositions; and providing additional health benefits that are synergistic to benefits and/or effects of administration of probiotics. The compositions of the present invention can be administered by any of a variety of known methods of administration, e.g., orally, dermatomucosally, (for example, dermally, sublingually, intranasally, and rectally), parenterally, (e.g. subcutaneous injection, intramuscular injection, intra-articular injection, intravenous injection), topically (transdermal) and by inhalation. Non-limiting examples of modes of administration include oral, transdermal, mucosal, sublingual, intramuscular, intravenous, intraperitoneal, subcutaneous, and combinations thereof.

“Loading Time Period”, as used herein means the period of time during which an initial maximum or loading dose of loading probiotic is administered to a user. The loading time period can be a defined length of time or can last until the user attains the desired benefits.

“Maintenance Time Period”, as used herein means the period of time during which a user feels consistent health benefits and wellbeing. A maintenance time period can continue throughout the life of a user.

“Pre-loading Time Period”, as used herein means the period of time before the loading time period.

The terms “Probiotic” and “Probiotics” as used herein can be used interchangeably and mean one or more natural, cultured, purified, genetically altered, and/or isolated strains of probiotic bacteria; products of probiotic bacteria; metabolites of probiotic bacteria; and mixtures, blends and combinations thereof. The probiotics of the present invention can be viable or non-viable when administered and/or when reaching the desired site of administration. The probiotics of the present invention can be administered together as a blend or mixture in a single dosage form, or can be administered in separate dosage forms at separate times.

As used herein, “Negative Adjustment Effects” associated with administration of probiotics include but are not limited to: altered sensations related to the passage of bowel movements, feelings of incomplete bowel movement, abdominal cramps, abdominal discomfort, abdominal distension, anxiety, belching, bloating, constipation, depression, diarrhea, disorders of motility, drowsiness, dyspepsia, erosive esophagitis, excess flatus, excessive drainage syndrome, feeling tired and weak, gastritis, gastroesophageal reflux, gastroparesis, headache, heartburn, hypersecretory conditions, indigestion, insomnia, irritability, itching, mechanical irritation of the bowel, nausea, nervousness, pain, rash, sleepiness, spasm, stomach problems, straining to have a bowel movement, sweating when having a bowel movement, urgency to have a bowel movement, vomiting, reactions to therapeutic compositions, and various combinations of these effects.

“User” as used herein is a mammal. Non-limiting examples of mammals with which the methods and kits of the present invention are useful include: humans and companion animals, non-limiting examples of which include: cats, dogs, guinea pigs, rabbits, ferrits, and horses.

Health problems, conditions, and diseases prevented or therapeutically managed by the methods and kits of the present invention include but are not limited to: abdominal cramps; abdominal discomfort; abdominal distension; antibiotic associated diarrhea (AAD); attention deficit/hyperactivity disorder; athralgias or other inflammatory conditions of the muscles or joint, autism spectrum disorders; autoimmune disorders particularly osteoarthritis, rheumatoid arthritis, lupus, multiple sclerosis and fibromyalgia; belching; bloating; cancer prevention, particularly colon cancer; celiac disease; cholecystitis; chronic pelvic pain syndrome; Clostridium difficile associated diarrhea (CDAD); Crohns disease; constipation (including chronic or functional constipation); dental caries or improved oral health; depression; diarrhea (including chronic or functional diarrhea, or traveler's diarrhea); disorders of immunoregulation or immunomodulation in particular as relating to food allergies, seasonal allergies, or environmental allergies, asthma, atopic dermatitis, eczema or other atopic disease; disorders of motility; diverticulitis; duodenal ulcers; dyspepsia (including functional dyspepsia); eating disorders such as anorexia and bulimia or other disorders of malnourishment; disorders of the skin; erosive esophagitis; excess flatus; feeling of incomplete bowel movement; feeling tired and weak; chronic fatigue syndrome; gall bladder disease; gastroesophageal reflux disease (GERD); gastroparesis; gastritis; gastric ulcers; halitosis; heartburn (including frequent heartburn); hypersecretory conditions such as Zollinger-Ellison syndrome; impaired mentation; impaired memory or other disorders of mental wellbeing; improved or modulation of gut-barrier function; improved overall health and wellbeing inflammatory bowel disease (IBD); irritable bowel syndrome (IBS) indigestion; lactose intolerance; mechanical irritation of the bowel; modulation or reduction of risk factors for cardiovascular disease; motion sickness; multiple endocrine adenomas; nausea; obesity; pain; prevention of osteoporosis posterior laryngitis; post-infection colitis; pouchitis; respiratory infection such as common cold; small intestine bacterial overgrowth (SIBO) or small bowel bacterial overgrowth (SBBO); spastic colon, spasm; stomach problems; straining to have a bowel movement; stress or altered stress responses such as somatization; systemic yeast, bacterial or viral infection including candiasis; systemic mastocytosis; sweating when having a bowel movement; ulcerative colitis (UC); urinary tract infection or cystitis, urgency to have a bowel movement; vaginosis and vaginitis viral diarrhea; visceral hypersensitivity, vomiting; reaction to therapeutic compositions and the like, or any combinations thereof.

### Methods

The present invention comprises methods of administering a probiotic including the steps of:
c. administering a loading dose of a loading probiotic for a loading time period; and
d. administering a dose of a botanical for the loading time period.

The present invention also comprises methods of administering a probiotic including the steps of:
c. administering a loading dose of a loading probiotic for a loading time period; and
d. administering a dose of an additional material for the loading time period.

The present invention comprises methods of administering a probiotic including the steps of:
a. administering a loading dose of a loading probiotic for a loading time period;
b. administering a dose of a botanical for the loading time period; and
c. administering a dose of an additional material for the loading time period.

The methods of the invention also include administering a maintenance dose of a maintenance probiotic for a maintenance time period; administering a dose of a botanical for a maintenance time period; administering a dose of an additional material for a maintenance time period; and combinations thereof.

The methods of the invention can also include optionally administering a dose of a pre-loading composition for a pre-loading time period.

The methods of the invention improve tolerability and perception of benefits of administering probiotics.

The invention also includes kits for use in administering probiotics; including loading doses of a loading probiotic to be administered for a loading time period; doses of a botanical to be administered for a loading time period; doses of an additional material to be administered for a loading time period; a compliance aid; instructions for use of the kit and the components thereof; and combinations thereof.

The kits can also include doses of a pre-loading composition to be administered for a pre-loading time period before the loading time period; doses of a probiotic to be administered for a maintenance time period; doses of a botanical to be administered for a maintenance time period; doses of an additional material to be administered for a maintenance time period; a compliance aid; instructions for use of the kit and the components thereof; and combinations thereof.

### Loading Time Period

The loading time period is comprised of either a predetermined time period or a time period sufficient to achieve alleviation of symptoms of the health problems, conditions, and diseases prevented or therapeutically managed by the methods and kits of the present invention.

Generally such a loading time period is from about 1 to about 60 days, alternatively from about 2 to about 55 days, alternatively from about 4 to about 40 days, and alternatively from about 7 to about 28 days. The determination of the loading time period can be aided by one or more compliance aids. During the loading time period, the amount and frequency of administration of the loading probiotic, botanical, and/or additional material can be adjusted as necessary during the loading time period based the user's symptoms and need for relief.

The loading probiotic, the botanical, and/or the additional material can be administered daily, every other day, every two days, or as often or seldom as desired to achieve alleviation of symptoms. The botanical and/or the additional material can be administered together with, or separately from, the loading probiotic, in the same or different dosage forms. The loading probiotic, botanical, and/or the additional material can be incorporated into a unit dosage form. The loading probiotic can be administered on the same day, at the same or different time(s) of day, as the botanical and/or the additional material, or can be administered on different days from the botanical and/or the additional material.

By way of non-limiting example, the loading probiotic can be administered in a cookie, a botanical in a tea, and a vitamin in a tablet, or they can be in the same cookie or capsule, or each in separate cookies or capsules or any other desired forms. According to the methods of the invention, the loading probiotic is administered at high loading doses initially during the loading time period to speed the effectiveness and relief of symptoms. The loading probiotic can be administered in a decreasing dosage over the loading time period, such that the dosage of loading probiotic at the end of the loading time period is appropriate for a continuing dosage of probiotic for a maintenance period. Alternatively, the loading probiotic can be administered at a loading dose for the duration of the loading time period, and following the loading time period, dosing of the probiotic can be discontinued as desired by a user.

### Loading Dosage Regimens

As used herein “loading dose” of the loading probiotic administered during the loading time period is an amount of loading probiotic that is higher than the normally recommended dose of the particular strain or strains of probiotic and is a dose effective to achieve alleviation of symptoms of the health problems, conditions, and/or diseases managed by the methods and kits of the present invention. The loading probiotic administered for the loading time period is administered at a loading dose concentration of from about 1×10³ to about 1×10¹⁴ colony forming units (cfu) of loading probiotic, alternatively from about 1×10⁵ to about 1×10¹⁴ cfu of loading probiotic, alternatively from about 1×10⁷ to about 1×10¹⁴ cfu of loading probiotic, alternatively from about 1×10⁹ to about 1×10¹² cfu of loading probiotic, and alternatively from about 1×10¹⁰ to about 1×10¹² cfu of loading probiotic, and alternatively from about 1×10¹¹ to about 1×10¹² cfu of loading probiotic, per day.

The loading dose can be administered in a single unit dose administered at any time during a day. Alternatively the loading dose can be administered in two or more doses administered at a single time of day or at two or more separate times of day.

The loading dose can be tapered from an initial high loading dose at the beginning of the loading time period to a lower dose at the end of the loading time period, on predetermined timing or when the user feels that his/her symptoms have been sufficiently alleviated and the user's body has adjusted to the loading probiotic. The dosage of loading probiotic can be tapered by the end of the loading time period to a dosage appropriate to maintain alleviation of symptoms during the maintenance time period. The administration of loading probiotic can be discontinued after the end of the loading time period, such that after the loading time period no probiotic is administered. The exact appropriate initial loading dosage amount will vary by individual user, based on the user's age, weight, condition or disease, and number, type and severity of symptoms.

The concentration of loading probiotic in a given dosage form and/or dose, and the total amount of loading probiotic delivered, either daily or per dose, will depend on the strain or strains of probiotic bacteria used. A loading dose of a given probiotic bacteria can be achieved by any of the following versus the normally recommended dose of the given probiotic bacteria: increasing the concentration of bacteria (i.e. increasing the amount of bacteria) in each dosage form administered, increasing the number of dosage forms given, increasing the frequency of dosage forms given, and combinations thereof.

By way of non-limiting example, an isolated strain of probiotic bacteria, Bifidobacterium infantis NCIMB 41003, can be administered at an initial loading dose of 3 capsules daily, each capsule containing 1×10⁹ cfu of bacteria, for a first week, 2 capsules daily, each capsule containing 1×10⁹ cfu of bacteria, for a second week, and 1 capsule daily, each capsule containing 1×10⁹ cfu of bacteria, for a third and a fourth week during a four week loading time period.

Alternatively, Bifidobacterium infantis NCIMB 41003 can be administered at an initial loading dose of one capsule daily containing 1×10¹² cfu of bacteria for a first two weeks, then one capsule daily containing 1×10⁹ cfu of bacteria for a second two weeks during a four week loading time period.

A botanical can be administered during all or a portion of the loading time period as desired by the user to achieve alleviation of negative adjustment effects of the loading probiotic; and/or alleviation of gastrointestinal and other health problems, conditions, diseases and associated symptoms. The amount and frequency of administration of the botanical can be adjusted as necessary during the loading time period based the user's symptoms and need for relief. The botanical can be administered daily, every other day, every two days, or as often or seldom as desired to achieve alleviation of symptoms. The botanical can be administered together with, or separately from, the loading probiotic, in the same or different dosage forms. The loading probiotic and botanical can be incorporated into a unit dose form. The botanical can be administered at the same or different time(s) of day as the loading probiotic, or on different days from the loading probiotic.

The botanical can be administered at therapeutic amounts and/or amounts recommended for general health, as would be understood by those of skill in the art. Such therapeutic and/or recommended amounts for general health can be found in a number of sources including by way of non-limiting example: The Physician's Desk Reference for Herbal Medicines (3^{rd} Ed.) Gruenwald, J., Thomson P D R, 2004; Natural Medicines Comprehensive Database, Jellin, J. M., 2005; The Complete German Commission E Monographs: Therapeutic Guide to Herbal Medicines, Blumenthal, M., Busse, W. R. Eds, Lippincott Williams & Wilkins, 1^{st} Ed., 1998; Quick Access Consumer Guide to Conditions, Herbs & Supplements, Anderson, R. A., 2000; and at the website of the U.S. National Institutes of Health at http://www.nlm.nih.gov/medlineplus/druginfo/herb All.html.

If a botanical is administered during the loading time period, the botanical can be administered at a dose of from about 0.001 mg to about 100 g, alternatively from about 0.01 g to about 50 g, alternatively from about 0.01 g to about 10 g, and alternatively from about 0.1 g to about 10 g of the botanical per day.

By way of non-limiting example, if the botanical is ginger, the ginger can be administered for the loading time period, at a dose of from about 10 mg (0.01 g) to about 10 g, and alternatively from about 1 g to about 5 g of ginger (Zingiber officinale) rhizome (root) or equivalent extract, tincture, oil, infusion, decoction, crystals or powder per day.

One or more additional materials selected from the group consisting of vitamins, minerals, metals, elements, essential fatty acids, essential amino acids, sensates, prebiotics, carotenoids, and combinations thereof can be administered during all or a portion of the loading time period as desired by the user to achieve alleviation of negative adjustment effects of the loading probiotic, and/or alleviation of other health problems, conditions, diseases and associated symptoms. The amount and frequency of administration of the additional material can be adjusted as necessary during the loading time period based the user's symptoms and need for relief. The additional material can be administered daily, every other day, every two days, or as often or seldom as desired to achieve alleviation of symptoms. The additional material can be administered together with, or separately from, the loading probiotic and/or botanical, in the same or different dosage forms. The loading probiotic and an additional material can be incorporated into a unit dose form. The additional material can be administered at the same or different time(s) of day as the loading probiotic and/or botanical, or on different days from the loading probiotic and/or botanical.

The additional material can be administered at therapeutic amounts and/or amounts recommended for general health, as would be understood by those of skill in the art. Such therapeutic and/or recommended amounts for general health can be found in a number of sources including by way of non-limiting example: The Physician's Desk Reference for Nutritional Supplements, Hendler, S. S., Rorvik, D. ed., Thomson Healthcare, 2001; The Physician's Desk Reference for Nonprescription Drugs and Dietary Supplements, ISSN: 1525-3678; A Guide to Understanding Dietary Supplements, Talbot, S. M., The Haworth Press, 2003; Quick Access Consumer Guide to Conditions, Herbs & Supplements, Anderson, R. A., 2000; at the website of the National Academy of Sciences, http://www.nap.edu; at the website of the U.S. Department of Agriculture at http://fnic.nal.usda.gov; and at the website of the Institute of Medicine of the National Academies at http://www.iom.edu.

If an additional material is administered during the loading time period, the additional material can be administered at a dose of from about 0.001 μg to about 10 g, alternatively from about 0.01 μg to about 5 g, and alternatively from about 0.1 μg to about 2 g of the additional material per day.

By way of non-limiting example, if a B Complex vitamin is administered daily for the loading time period a dose (for example, 1 tablet) could contain from about 0.3 mg to about 1000 mg of Vitamin B1 (thiamin or thiamine), from about 0.4 mg to about 500 mg of Vitamin B2 (riboflavin), from about 6 mg to about 2000 mg of Vitamin B3 (niacin, niacinamide or nicotinic acid), from about 2.5 mg to about 20,000 mg of Vitamin B5 (pantothenic acid), from about 0.3 mg to about 1000 mg of Vitamin B6 (pyridoxine), from about 35 μg to 15 mg of Vitamin B7 (biotin), 30 μg to 20 mg of Vitamin B9 (folic acid, folinic acid, or folate), and 0.5 μg to 10 mg of Vitamin B12 (cobolamine, cyanocobalamin, hydroxycobalamin, methylcobalamin). A non-limiting example of a B Complex vitamin is Stress B-Complex available from Nature Made Nutritional Products, Mission Hills, Calif., USA.

### Maintenance Time Period

The maintenance time period can begin at a predetermined time period or can begin when the user feels that his/her symptoms have been sufficiently alleviated. The user can track his/her progress and feeling using a compliance aid such as a diary, chart, graph, color coded tracker, or combination thereof, such as that described in pending U.S. patent application Ser. No. 11/319,839, alone or in combination with using color coded compliance aid devices for dosing as described below.

The maintenance time period can continue throughout the life of the user. The user can administer a dose of a maintenance probiotic throughout the user's life to maintain desired gastrointestinal function, desired health benefits, to prevent and/or maintain achieved alleviation of symptoms of negative adjustment effects of administration of the maintenance probiotic, and/or symptoms of health problems, conditions and/or diseases managed by the present invention. The dose of maintenance probiotic administered for the maintenance time period can be less than the loading dose of loading probiotic administered during the loading time period, and can be a dose sufficient to maintain alleviation of symptoms.

### Maintenance Dosage Regimens

The maintenance probiotic administered during the maintenance time period is administered in an amount effective to maintain alleviation of symptoms, and/or maintain a feeling of wellbeing. The maintenance probiotic administered for the maintenance time period is administered at a bacteria concentration of from about 1×10³ to about 1×10¹² colony forming units (cfu) of maintenance probiotic, alternatively from about 1×10⁵ to about 1×10¹² cfu of maintenance probiotic, and alternatively from about 1×10⁷ to about 1×10¹² cfu of maintenance probiotic per day.

The maintenance probiotic can be administered in a single unit dose administered at any time during a day. Alternatively the maintenance probiotic can be administered in two or more doses administered at a single time of day or at two or more separate times of day. During the maintenance time period, a maintenance probiotic can be administered at various times of day, can be administered every day, every other day, every two days, or at whatever interval is desired by the user and effective to maintain desired feeling of wellbeing, including having no symptoms, very few symptoms, very mild symptoms, and combinations thereof. The dosage amount and frequency can be varied according to the presence or absence of symptoms, as desired by the user. If symptoms worsen or recur, the dosage amount and/or frequency of the maintenance probiotic can be adjusted as needed to alleviate symptoms. The maintenance probiotic can be the same probiotic as the loading probiotic, a different probiotic, and/or a mixture of probiotics.

A botanical can optionally be administered during the maintenance time period. The botanical can be administered during all or a portion of the maintenance time period as desired to maintain alleviation of symptoms of negative adjustment effects related to administration of the maintenance probiotic, and/or to maintain alleviation of gastrointestinal and/or other health problems, conditions, diseases and associated symptoms. If a botanical is administered during the maintenance time period, the botanical can be the same or a different botanical than that administered during the loading time period. The amount and frequency of administration of the botanical can be adjusted as needed. The botanical can be administered daily, every other day, every two days, or as often or seldom as desired to maintain alleviation of symptoms. If administered during the maintenance time period, the botanical can be administered together with, or separately from, the maintenance probiotic in the same or different dosage forms. The maintenance probiotic and botanical can be incorporated into a unit dosage form. The botanical can be administered at the same or different time(s) of day as the maintenance probiotic, or on different days from the maintenance probiotic.

If a botanical is administered during the maintenance time period, the botanical can be administered at a dose of from about 0.001 mg to about 100 g, alternatively from about 0.01 g to about 50 g, alternatively from about 0.01 g to about 10 g, and alternatively from about 0.1 g to about 10 g of the botanical per day.

By way of non-limiting example, if the botanical is ginger, the ginger can be administered for the maintenance time period, at a dose of from about 1 mg to about 10 g, and alternatively from about 1 g to about 5 g of ginger (Zingiber officinale) rhizome (root) or equivalent extract, tincture, oil, infusion, decoction, crystals or powder per day.

One or more additional materials selected from the group consisting of vitamins, minerals, metals, elements, essential fatty acids, essential amino acids, sensates, prebiotics, carotenoids, and combinations thereof can be administered during all or a portion of the maintenance time period as desired to maintain alleviation of negative adjustment effects related to administration of the maintenance probiotic, and/or alleviation of symptoms of gastrointestinal and/or other health problems, conditions, diseases and associated symptoms.

If one or more additional materials is administered during the maintenance time period, the additional material can be the same or different than that administered during the loading time period. The amount and frequency of administration can be adjusted as needed. The additional material can be administered daily, every other day, every two days, or as often or seldom as desired to maintain alleviation of symptoms. If administered during the maintenance time period, the additional material can be administered together with, or separately from, the maintenance probiotic and/or botanical, in the same or different dosage forms. The additional material can be administered at the same or different time(s) of day as the maintenance probiotic and/or botanical, or on different days from the maintenance probiotic and/or botanical.

If an additional material is administered during the maintenance time period, the additional material can be administered at a dose of from about 0.001 μg to about 10 g, alternatively from about 0.01 g to about 5 g, and alternatively from about 0.1 μg to about 2 g of the additional material per day.

By way of non-limiting example, if a B Complex vitamin is administered daily for the loading time period a dose (for example, 1 tablet) could contain from about 0.3 mg to about 1000 mg of Vitamin B1 (thiamin or thiamine), from about 0.4 mg to about 500 mg of Vitamin B2 (riboflavin), from about 6 mg to about 2000 mg of Vitamin B3 (niacin, niacinamide or nicotinic acid), from about 2.5 mg to about 20,000 mg of Vitamin B5 (pantothenic acid), from about 0.3 mg to about 1000 mg of Vitamin B6 (pyridoxine), from about 35 μg to 15 mg of Vitamin B7 (biotin), 30 μg to 20 mg of Vitamin B9 (folic acid, folinic acid, or folate), and 0.5 μg to 10 mg of Vitamin B12 (cobolamine, cyanocobalamin, hydroxycobalamin, methylcobalamin). A non-limiting example of such a B Complex vitamin is Stress B-Complex available from Nature Made Nutritional Products, Mission Hills, Calif., USA.

### Pre-Loading Time Period

The methods of the present invention can also include administering a pre-loading composition. The pre-loading composition can be administered for a pre-loading time period. The pre-loading time period occurs immediately before the loading time period. The pre-loading time period can be from about 1 to about 60 days in duration, and the loading time period begins immediately following the end of the pre-loading period. However, administration of the pre-loading composition can continue through the loading time period and the maintenance time period. The pre-loading time period simply begins before the loading time period.

### Pre-Loading Dosage Regimens

The pre-loading composition, administered beginning at the pre-loading time period, and optionally continuing throughout the loading time period and/or the maintenance time period, can be administered in varying amounts depending on the type of pre-loading composition used, and whether a pre-loading composition is used. The pre-loading composition can be administered as a single unit dose, for example once per day, or can be administered in multiple doses multiple times daily. The pre-loading composition can be administered daily, once every other day, once every two days, or as often or seldom as desired to allow the user's body to adjust to the pre-loading composition. The pre-loading composition is selected from the group consisting of probiotics, botanicals, additional materials, and combinations thereof.

### Compositions

### Probiotic Compositions

The probiotic(s) used in the methods and kits of the present invention can be any beneficial symbiotic bacteria. Probiotic bacteria of the present invention can be bacteria that are indigenous and normal inhabitants of natural soils and freshwater, those found in organically grown fruits and vegetables, milk, and considered non-toxic and non-pathogenic. Particularly, the probiotic bacteria as used in the present invention are bacteria of human and/or animal origin. As used in the present invention, the probiotic can be viable or non-viable. The probiotic can restore the balance of bacteria in the gastrointestinal tract or other body system, thus helping to prevent and/or alleviate the problems, diseases, conditions and symptoms managed by the present invention. Furthermore, one or more different, viable and/or non-viable, probiotics can be used in the methods and kits of the present invention.

By way of non-limiting example, a loading probiotic can be administered during the loading time period, and the same or a different probiotic can be administered as the maintenance probiotic during the maintenance time period. A third probiotic can optionally be administered during the pre-loading time period. Alternatively, the same probiotic can be administered for the pre-loading and loading time periods, and a different probiotic can be administered for the maintenance time period. Alternatively, a first probiotic can be administered during the pre-loading time period, and a second probiotic can be administered during the loading and maintenance time periods. Alternatively, blends, mixtures, and/or combinations of probiotics can be administered in the pre-loading, loading and/or the maintenance time periods.

Non-limiting examples of probiotics useful with the present invention include bacteria selected from the group consisting of Bifidobacterium, Lactobacillus, and Streptococcus. Particular non-limiting examples of probiotics useful herein include Lactobacillus planetarium, Lactobacillus salivarius, Lactobacillus rueteri, Lactobacillus bulgaricus, Lactobacillus casei, Lactobacillus rhamnosus, Lactobacillus sporogenes, Lactococcus lactis, Biffidophilus infantis, Streptococcus thermophilous, Bifodophilus longum, Bifidobacteria bifidus, Arthrobacter agilis, Arthrobacter citreus, Arthrobacter globiformis, Arthrobacter leuteus, Arthrobacter simplex, Azotobacter chroococcum, Azotobacter paspali, Azospirillum brasiliencise, Azospriliium lipoferum, Bacillus brevis, Bacillus macerans, Bacillus pumilus, Bacillus polymyxa, Bacillus subtilis, Bacteroides lipolyticum, Bacteroides succinogenes, Brevibacterium lipolyticum, Brevibacterium stationis, Kurtha zopfil, Myrothecium verrucaris, Pseudomonas calcis, Pseudomonas dentrificans, Pseudomonas flourescens, Pseudomonas glathei, Phanerochaete chrysosporium, Streptmyces fradiae, Streptomyces cellulosae, Stretpomyces griseoflavus, Bacillus laterosporus, Bacillus bifidum, Bacillus laterosporus, and combinations thereof.

In certain embodiments of the present invention, a purified, isolated, and/or genetically altered bacterial strain can be used. Such a strain can be genetically altered in any of a variety of different ways to increase efficacy and/or effectiveness. Exemplary methods are described in Methods in Cloning Vol. 3, eds. Sambrook and Russell, Cold Spring Harbor Laboratory Press (2001) and references cited therein. In addition, probiotic bacteria of the present invention can be obtained by any available means. A variety of beneficial bacteria are commercially available from American Type Culture Collection Catalogue (Rockville, Md.). Beneficial bacteria can also be cultured, for example, in liquid, or on solid media, following routine and established protocols, and isolated from the medium by any available means. Exemplary methods are described in Methods in Cloning Vol. 3, eds. Sambrook and Russell, Cold Spring Harbor Laboratory Press (2001) and references cited therein.

As a non-limiting example, strains of Bifidobacterium isolated from resected and washed human gastrointestinal tract as disclosed in WO 00/42168 can be used. An example includes Bifidobacterium infantis strain designated UCC35624, described as being deposited at the National Collections of Industrial and Marine Bacteria Ltd (NCIMB) on Jan. 13, 1999, and accorded the accession number NCIMB 41003. The Bifidobacterium infantis disclosed herein is described, for example, in issued U.S. Pat. No. 7,195,906.

### Botanicals

The botanical of the methods and kits of the present invention exert beneficial effects on the gastrointestinal tract, including soothing or demulcent effects, gas reducing or carminative effects, anti-diarrheal or astringent effects, laxative or aperient, cathartic, purgative or hydrogogue effects, analgesic, antispasmodic or relaxation effects, stimulant or bitter effects, or acts as a digestive and health aid. The botanical may also exert beneficial effects on areas of the body other than the gastrointestinal tract, as exemplified by a reduction of drowsiness, fatigue, headache, boosting of immune response, and the like.

The botanical particularly aids in reducing the unpleasant negative adjustment effects that often are perceived to accompany initial administration of a probiotic. The unpleasant adjustment effects often lead to lack of user compliance, and limit the amount of probiotic that can be initially introduced. However, initial low dosing of probiotic leads to increased time to effectiveness of the probiotic. In contrast, the addition of a botanical to the methods and kits of the present invention enables high loading doses of probiotic initially, by reducing the unpleasant negative adjustment effects that would normally prohibit high initial loading dosing of probiotic. Thus, the present invention provides a reduced time to perceived effectiveness, and increased user compliance, by allowing high initial loading doses of probiotic by reducing unpleasant adjustment effects thereof. The botanical can also provide overall digestive and wellness benefits.

By way of non-limiting example, a botanical can be administered during the loading time period, and the same or a different botanical can be administered during the maintenance time period. A third botanical can optionally be administered during the pre-loading time period. Alternatively, the same botanical can be administered for the pre-loading and loading time periods, and a different botanical can be administered for the maintenance time period. Alternatively, a first botanical can be administered during the pre-loading time period, and a second botanical can be administered during the loading and maintenance time periods. Alternatively, blends, mixtures, and/or combinations of botanicals can be administered in the pre-loading, loading and/or the maintenance time periods.

Non-limiting example of botanicals useful in the methods and kits of the present invention include the ginger Family (Zigiberaceae); licorice root (Glycyrrhizin glabra); marshmallow root (Althea officinalis, Althea radix); Chamomile (Matricariae flos, Chamaemelum nobile); Fennel oil, Fennel Seed (Foeniculum vulgare); Caraway oil, Caraway seed (Carum carvi, Carvi fructus, Carvi aetheroleum); Lemon Balm (Melissae folium, Melissa); Horehound Herb (Murrubii herba); Flaxseed, flaxseed alpha-linoleic acid (Lini semen); Rosemary Leaf, rosemary extract (Rosmarinus officinalis, Rosemary folium); polyphenols, avocado extract comprising mannoheptulose, mannoheptulose (Persea Americana), and combinations thereof.

Botanicals from the ginger Family (Zigiberaceae) are particularly useful. Non-limiting examples of botanicals from the ginger Family include Aframomum chrysanthum (aframomum), Aframomum citratum (Mbongo), Aframomum melegueta (Grains of paradise), Alpinia formosana (pinstripe ginger), Alpinia galanga (Greater galanga), Alpinia japonica kinisiana (‘Peppermint Stick)’ (alpinia), Alpinia officinarum (galangal), Alpinia purpurata ‘Pink Ginger’ (pink ginger), Alpinia purpurata ‘Red Ginger’ (red ginger), Alpinia purpurata ‘Anne Hironaka’ (white ginger), Alpinia purpurata ‘Polynesian Princess’ (candy cane ginger), Alpinia purpurata ‘Rosy Dawn’ (pink ginger), Alpinia purpurata ‘Tahitian Ginger’ (double red ginger), Alpinia zerumbet (shell ginger), Alpinia zerumbet ‘Yu Hwa’ (Chinese variegated ginger), Alpinia zerumbet ‘Variegata’ (variegated shell ginger), Amomum subulatum (Black Cardamom), Boesenbergia rotunda, Boesengergia pandurata (Fingerroot), Costus varzearum (costus), Cucuma cordata ‘Jewel of Thailand’ (curcuma), Cucuma flaviflora ‘Red Fireball’ (curcuma), Curcuma elata (rose turmeric), Curcuma longa (C. domestica) (turmeric), Curcuma ornata (curcuma), Curcuma parviflora (curcuma), Curcuma petiolata (hidden lily), Curcuma petiolata ‘Emperor’ (curcuma), Curcuma roscoeana (jewel of Burma), Curcuma sp. ‘Figi’ (curcuma), Curcuma sp. ‘Nardo’ (curcuma), Curcuma sp. ‘Purple Gusher’ (curcuma), Curcuma sp. ‘Siam Princess’ (curcuma), Curcuma zedoaria (curcuma), Elettaria cardamomum (Green cardamom), Etlingera corneri ‘Rose of Siam’ (ginger), Etlingera elatior ‘Alii Chang’ (pink spider torch ginger), Etlingera elatior ‘Pink Torch’ (pink torch ginger), Etlingera elatior ‘Red Torch’ (red torch ginger), Etlingera elatior ‘Tulip Torch’ (tulip torch ginger), Etlingera elatior ‘White Torch’ (white torch ginger), Etlingera fulgens (burgundy tulip ginger), Etlingera newmanii, Etlingera venusta (Malay rose), Globba pendula ‘Silver Comet’ (silver globba), Globba patens, Globba winitii (purple globba), Hedychium angustifolium ‘Peach’ (hedychium), Hedychium coccineum ‘Disney’ (hedychium), Hedychium coronarium (white ginger), Hedychium coronata ‘Crema’, Hedychium ellipticum (hedychium), Hedychium flavescens (cream ginger), Hedychium gardnerianum (kahili ginger), Hedychium greenei (red leaf ginger), Hedychium sp. ‘Ayo’ (hedychium), Hedychium sp. ‘Brandie Saito’ (hedychium), Hedychium sp. ‘Carnival’ (hedychium), Hedychium sp. ‘Dr. Moy’ (variegated hedychium), Hedychium sp. ‘Elizabeth’ (hedychium), Hedychium sp. ‘Filagree’ (hedychium), Hedychium sp. ‘Gold Flame’ (hedychium), Hedychium sp. ‘Kinkaku’ (hedychium), Hedychium sp. ‘Luna Moth’ (hedychium), Hedychium sp. ‘Maiko’ (hedychium), Hedychium sp. ‘Multiflora White’ (hedychium), Hedychium sp. ‘Pale Yellow’ (hedychium), Hedychium sp. ‘Pink Flame’ (hedychium), Hedychium sp. ‘Pink Sparks’ (hedychium), Hedychium sp. ‘Pink V’ (hedychium), Hedychium sp. ‘Pradhanii’ (hedychium), Hedychium sp. ‘Sherry Baby’ (hedychium), Hedychium sp. ‘Shurei’ (hedychium), Hedychium sp. ‘Tropic Bird’ (hedychium), Hedychium thrysiforme (hedychium), Kaempferia galanga (Lesser galanga), Kaempferia rotunda (Asian crocus), Kaempferia roscoeana, Mantisia salitoria, Renealmia occidentalis (red renealmia), Riedelia coralina (pink riedelia), Smithiatris supraneeani, Tapinocheilos ananasae, Zingiber gramineum, Zingiber mioga ‘Dancing Crane’ (variegated zingiber), Zingiber newmanii (ginger), Zingiber sp. ‘Chocolate Shampoo’ (ginger) Zingiber officinale (Ginger), and combinations thereof.

### Additional Materials

Additional materials useful in the present invention are selected from the group consisting of vitamins, minerals, metals, elements, essential fatty acids, essential amino acids, sensates, prebiotics, carotenoids, and combinations thereof. The additional materials of the methods and kits of the present invention exert beneficial effects synergistic to those of the probiotic bacteria and/or botanical.

The additional materials can aid in reducing unpleasant negative adjustment effects that often are perceived to accompany initial administration of a probiotic. The unpleasant adjustment effects often lead to lack of user compliance, and limit the amount of probiotic that can be initially introduced. However, initial low dosing of probiotic leads to increased time to effectiveness. In contrast, the addition of one or more additional materials to the methods and kits of the present invention enables high loading doses of probiotic initially, by reducing the unpleasant negative adjustment effects that would normally prohibit high initial loading dosing of probiotic. Thus, the present invention provides a reduced time to perceived effectiveness, and increased user compliance, by allowing high initial doses of probiotic by reducing unpleasant adjustment effects. The additional materials can also provide overall digestive and wellness benefits, non-limiting examples of which include increased energy.

By way of non-limiting example, one or more additional materials can be administered during the loading time period, and the same or a different additional material can be administered during the maintenance time period. A third additional material can optionally be administered during the pre-loading time period. Alternatively, the same additional material can be administered for the pre-loading and loading time periods, and a different additional material can be administered for the maintenance time period. Alternatively, a first additional material can be administered during the pre-loading time period, and a second additional material can be administered during the loading and maintenance time periods. Alternatively, blends, mixtures and/or combinations of additional materials can be administered in the pre-loading, loading and/or the maintenance time periods.

### Non-Limiting Examples of Vitamins Include:

Vitamin A (retinoids (retinol, retinoids, carotenoids)), Vitamin B1 (thiamine or thiamin), Vitamin B2 (riboflavin), Vitamin B3 (niacin, niacinamide, nicotinic acid), vitamin B5 (pantothenic acid), Vitamin B6 (pyridoxine); Vitamin B7 (biotin), Vitamin B9 (folic acid, folinic acid, folate), Vitamin B12 (cobolamine, cyanocobalamin, hydroxycobalamin, methylcobalamin), Vitamin C (ascorbic acid), Vitamin D (ergocalciferol, cholecalciferol), Vitamin E (tocopherols, tocotrienols), Vitamin K (phylloquinone, menaquinones), and combinations thereof.

### Non-Limiting Examples of Minerals, Metals, and Elements (and Physiologically Acceptable Salts Thereof) Include:

Calcium (Calcium phosphate, Calcium glubionate, Calcium gluconate, Calcium carbonate, Calcium lactate, Calcium lactate gluconate, Calcium chloride, Calcium glycerylphosphate, Calcium citrate lysine complex, Calcium glucoheptonate, Calcium pangamate), Potassium (Potassium chloride, Potassium citrate, Potassium hydrogentartrate, Potassium hydrogencarbonate, Potassium gluconate), Sodium (Sodium chloride, Sodium sulfate), Zinc (Zinc sulfate, Zinc gluconate), Magnesium (Magnesium chloride, Magnesium sulfate, Magnesium gluconate, Magnesium citrate, Magnesium aspartate, Magnesium lactate, Magnesium levulinate, Magnesium pidolate, Magnesium orotate, Magnesium oxide), Fluoride (Sodium fluoride, Sodium monofluorophosphate), Selenium (Sodium selenate, Sodium selenite); Iron, Iodine, Copper, Boron, Fluorine, Chromium, Silicon, and combinations thereof.

### Non-Limiting Examples of Essential Fatty Acids Include:

Linolenic acid, Linoleic acid, and combinations thereof.

### Non-Limiting Examples of Essential Amino Acids Include:

Alanine, Cysteine, Aspartic acid, Glutamic acid, Phenylalanine, Glycine, Histidine, Isoleucine, Lysine, Leucine, Methionine, Asparagine, Proline, Glutamine, Arginine, Serine, Threonine, Valine, Tryptophan, Tyrosine, and combinations thereof.

B vitamins are particularly useful. Non-limiting examples include combinations of Vitamins B1, B2, niacin, pantothenic acid, B6, biotin, folic acid, and B12.

The methods and kits of the present invention can also comprise an additional material that creates a sensorial experience that can provide an early signal and/or perception of relief and/or efficacy. Such an additional material can be a called a sensate. By “sensate” is meant a compound or composition that is perceived by a sense or the senses, or has a physical sensation. Such an ingredient can be used to enhance the perception of the benefits of the compositions used in the methods and kits of the present invention. Alternatively, a sensate can act as a counter-stimulant or counter-irritant i.e. by creating an alternate sensation that diverts attention from any untoward effects via reflex action of the sense (taste, smell, etc.) stimulated by the sensate.

Non-limiting examples of sensates useful in the methods and kits of the present invention include: peppermint, vanilla, spearmint, warming agents, cooling agents, bitter agents, tingling agents, and combinations thereof as would be known to those of skill in the art. A non-limiting example of use of such a sensate can be in a tablet coated with a cooling compound that creates a soothing sensation upon swallowing, and/or a continued cooling effect as it moves down the esophagus, and/or a continued cooling and/or soothing effect after swallowing. By way of non-limiting example, lesser amounts of sensate can be used for immediate action localized to the mouth and/or throat area, whereas greater amounts of sensate can be used for action in the mouth, throat, esophagus, stomach and further along the digestive tract.

The additional materials of the methods and kits of the present invention can also comprise a prebiotic, non-limiting examples of which include: bifidogenic compounds, lactogenic compounds, and combinations thereof. “Bifidogenic” and “Lactogenic” as used herein mean resulting in selective stimulation of the growth activity of probiotic bacteria including but not limited to bifidobacteria and lactobacteria.

Non-limiting examples of such bifidogenic and lactogenic prebiotic compounds include: fructo-oligosaccharides (FOS), oligofructose, fructans including inulin and levan, isomalto-oligosaccharides including isomaltose, panose, isomaltotetraose, isomaltopentaose, nigerose, kojibiose, and isopanose, trans-galacto-oligosaccharides, soy oligosaccharides including raffinose and stachyose, xylo-oligosaccharides, manno-oligosaccharides, lactulose, lactilol, lactosucrose, pyrodextrins, fiber gums including acacia, carrageenan, guar gum, locust bean gum, xanthan gum, resistant starch (i.e. starch resistant to digestion in the stomach and small intestine), and combinations thereof.

A “carotenoid” is a class of pigments occurring in the tissues of higher plants, algae, bacteria and fungi. Non-limiting examples of carotenoids include: lutein, astaxanthin, zeaxanthin, bixin, lycopene, beta-carotene and mixtures and/or combinations thereof.

### Pre-Loading Composition

The pre-loading composition can be used to prepare a user's system for introduction of the loading dose of loading probiotic at the beginning of the loading time period. The pre-loading composition can also be used to help reduce any initial unpleasant negative adjustment effects perceived by the user upon introduction of the loading probiotic at the beginning of the loading time period. In addition, the pre-loading composition can aid in preparing a beneficial environment for the subsequent delivery of the loading dose of the loading probiotic, earlier effectiveness of relief of symptoms, and/or perceived earlier effectiveness of relief of symptoms of the health problems, conditions and/or diseases treated and/or prevented by the present invention.

Non-limiting examples of a pre-loading composition used in the methods and kits of the present invention include a probiotic, a botanical, an additional material, and combinations thereof. Useful probiotics, botanicals, and additional materials are described above.

### Dosage Forms

The probiotic, botanical, additional material, and combinations thereof can be administered separately in separate dosage forms. The probiotic, botanical, additional material, and combinations thereof can be administered separately or together, in the same or different dosage form and/or in any combination thereof.

Non-limiting examples of dosage forms of into which the probiotic, botanical, additional material and combinations thereof can be incorporated include capsule, chewable tablet, swallowable tablet/pill, buccal tablet, coated tablet, troche, powder, lozenge, soft chew, solution, suspension, spray, extract, tincture, oil, decoction, infusion, syrup, elixir, wafer, food product, and combinations thereof.

The dosage forms can comprise ingestable carriers, non-limiting examples of which include solid or liquid filler diluents, encapsulating substances, and mixtures and combinations thereof; sugars; starches; cellulose and its derivatives; powdered tragacanth; malt; gelatin; talc; stearic acid; magnesium stearate; vegetable oils; polyols; agar; alginic acid; pyrogen-free water; isotonic saline; phosphate buffer solutions; wetting agents; lubricants; coloring agents; flavoring agents; preservatives; and combinations thereof.

Non-limiting examples of food products include acidified milk, yogurt, milk powder, tea, juice, beverage, confection (which herein includes candies and chocolates), chewable bar, cookie, wafer, cracker, cereal, soft chew, treat, and combinations thereof.

Non-limiting examples of dosage forms of botanicals particularly suited to the methods and kits of the present invention include extract, tincture, oil, fresh or dried root or rhizome, infusion or decoction, powdered root or rhizome, whole root or rhizome, crystallized matter and combinations thereof. Such dosage forms of botanical can be incorporated into other dosage forms of the present invention, i.e. an extract can be incorporated into a capsule or infusion.

By way of non-limiting example, the pre-loading composition can be administered as a chewable tablet coated with a sensate, a botanical can be administered as an infusion, one or more vitamins, minerals, metals, elements, essential fatty acids, essential amino acids, prebiotics, and combinations thereof can be administered as a swallowable pill, and the loading probiotic can be administered in a capsule. Alternatively, the loading probiotic, botanical, and/or additional material can be administered in a single swallowable capsule.

### Method of Making

Preferred oral dosage forms of the present invention may be prepared by any known or otherwise effective techniques known in the art that are suitable to provide final product forms of capsule, chewable tablet, swallowable tablet/pill, buccal tablet, coated tablet, troche, powder, lozenge, soft chew, solution, suspension, spray, extract, tincture, oil, decoction, infusion, syrup, elixir, wafer, food product such as acidified milk, yogurt, milk powder, tea, juice, beverage, confection (which includes candies and chocolates), chewable bar, cookie, wafer, cracker, cereal, treat, and combinations thereof, for oral ingestion and absorption to prevent or treat gastrointestinal diseases, conditions, symptoms and/or provide health benefits.

### Kits

The kits of the present invention can be used in administering probiotics and can comprise:
a. loading doses of a loading probiotic to be administered for a loading time period; and components selected from:
b. doses of a botanical to be administered for a loading time period;
c. doses of an additional material to be administered for a loading time period;
d. instructions for use of the kit; and
e. a compliance aid.

The kits can also comprise maintenance doses of a maintenance probiotic to be administered for a maintenance time period; doses of a botanical to be administered for a maintenance time period; doses of an additional material to be administered for a maintenance time period; instructions for use of the kit; a compliance aid; and combinations thereof.

The kits can also comprise pre-loading composition to be administered for a pre-loading time period.

Separate kits for a pre-loading time period, loading time period, and maintenance time period can be provided. Alternatively, the kits can comprise a combination of components for pre-loading, loading, and maintenance time periods in the same kit.

### Instructions

The instructions can include direction which can be oral direction (e.g., through oral instruction from, for example, a physician, veterinarian, health professional, sales professional or organization, and/or radio or television media (i.e., advertisement) or written direction (e.g., through written direction from, for example, a physician, veterinarian or other health professional (e.g., scripts), sales professional or organization (e.g., through, for example, marketing brochures, pamphlets, or other instructive paraphernalia), written media (e.g., internet, electronic mail, or other computer-related media), and/or devices associated with the composition (e.g., a label present on a package containing the composition). The instructions can be provided, contained, stored, and/or delivered in a variety of forms including, for example, paper, computer, personal digital assistant, telephone (including cellular phone and other communication devices), BLACKBERRY® or other devices used for communicating voice or text), Internet, and the like.

### Compliance Aid

The kits of the present invention can also include one or more compliance aids for facilitating compliance and/of allowing the user to visually track progress. Non-limiting examples of a compliance aid which can be used to track progress include a diary, chart, fillable color coded chart, and tracking device, and combinations thereof. The compliance aid can be provided, contained, stores, and/or delivered in a variety of forms including, for example, paper, computer, personal digital assistant, telephone (including cellular phone and other communication devices), BLACKBERRY® or other devices uses for communicating voice or text, Internet, and the like. A compliance aid useful with the methods of the present invention is described in U.S. patent application Ser. No. 11/391,839.

Another form of compliance aid can be a dosing or packaging device. Such a compliance aid can include various color coded dosing devices that aid the user in identifying which dosages to take on what day at what time, thereby facilitating dosing and compliance. Non-limiting examples of such dosing devices include blister cards, blister packs and/or other forms of device for containing dosing forms. The devices can have different colors and/or shades of colors to denote different days of the week, different times of day, dosage amount, composition to be administered, i.e. loading probiotic, botanical, additional material, maintenance probiotic, pre-loading composition, and combinations thereof. There can also be text identifying dosing order, day, time of day, dosage amount, composition to be administered, and combinations thereof. In the methods of the invention, one or more blister packs can be supplied in order to aid a user in compliance with the methods of the invention.

A form of device useful as a compliance aid with the methods of the present invention can be a blister pack. Blister packs are well described in the art as commonly used unit-dose packaging for medicinal products, in particular tablets, capsules or lozenges. Blister packs are the main packaging type for products where pharmacy dispensing and repackaging are not common. A series of blister cavities is sometimes called a blister card or blister strip, or alternately, a blister pack. In some parts of the world a blister pack is known as a Push-Through-Pack (PTP). The main advantages of this type of packaging are the assurance of product and packaging integrity, including shelf life of each individual dose, and the possibility of creating a compliance pack or calendar pack.

Blister packs are generally created by means of a form-fill seal process. A form-fill-seal process means that the blister pack is created from rolls of flat sheet or film, filled with the pharmaceutical product and closed (sealed) on the same equipment. Such equipment is called a blister line.

Blister packs comprise two principle components: 1) a formed base web creating a cavity inside which a product fits and 2) a lidding foil for dispensing the product out of the pack. There are 2 ways of forming the cavity into a base web sheet: thermoforming and cold forming.

In the case of thermoforming, a plastic film or sheet is unwound from a reel and guided through a pre-heating station on the blister line. The temperature of the pre-heating plates (upper and lower plates) is such that the plastic will soften and become moldable. The warm plastic will then arrive in a forming station where a large pressure (4 to 8 Bar) will form the blister cavity into a negative mold. The mold is cooled such that the plastic becomes rigid again and maintains its shape when removed from the mold. In case of difficult shapes, the warm film will be physically pushed down partially into the cavity by a “plug-assist” feature.

In the case of cold forming, an aluminum based laminate film is simply pressed into a mold by means of a stamp. The aluminum will be elongated and maintain the formed shape. In the industry these cold formed blisters are called Cold Form Foil (CFF) blisters. The principal advantage of Cold Form Foil blisters is that the use of aluminum provides a near complete barrier for water and oxygen, allowing an extended product expiry date. The principal disadvantages of Cold Form Foil blisters are: slower speed of production compared to thermoforming; lack of transparency of the package (a therapy compliance disadvantage); and a larger size of blister pack (aluminum can not be formed with near 90 degree angles) vs thermoformed blister packs.

### Figures

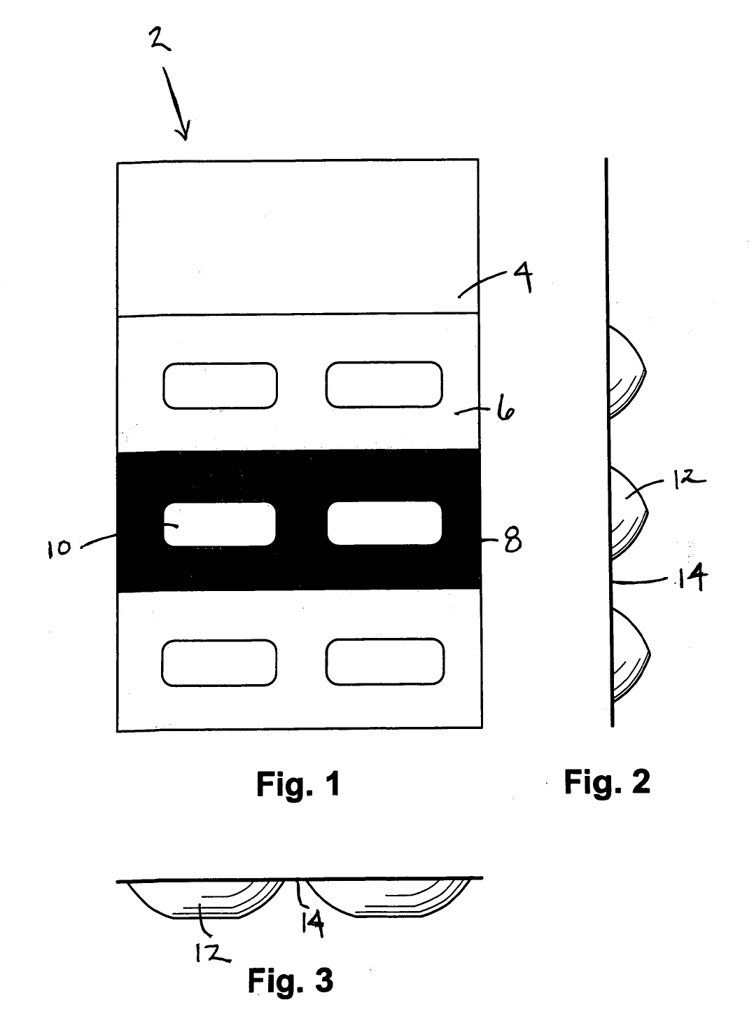

FIGS. 1-3 illustrate an embodiment of a color coded compliance aid of the present invention having two shades of color for different times of day and/or different days. FIG. 1 is a top plan view of a blister pack compliance aid 2 showing the top 4 of the blister pack 2 having alternating light 6 and medium 8 color shading regions, and having six windows 20 through which a capsule or tablet can be pushed to retrieve the capsule or tablet. The shading can be of any color and/or colors to differentiate dosing and can be accompanied by text to enhance clarity of dosing. FIG. 2 is a right side view thereof, showing cavities 12 on the bottom side 14. Cavities 12 would contain capsules or tablets. FIG. 3 is a front view thereof, showing cavities 12.

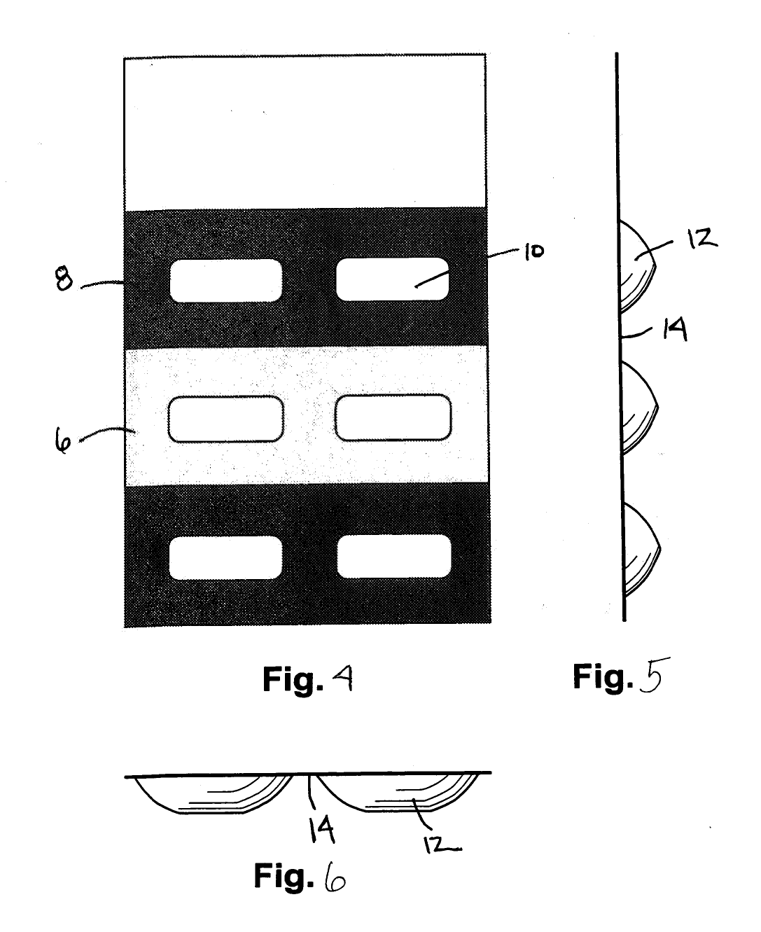

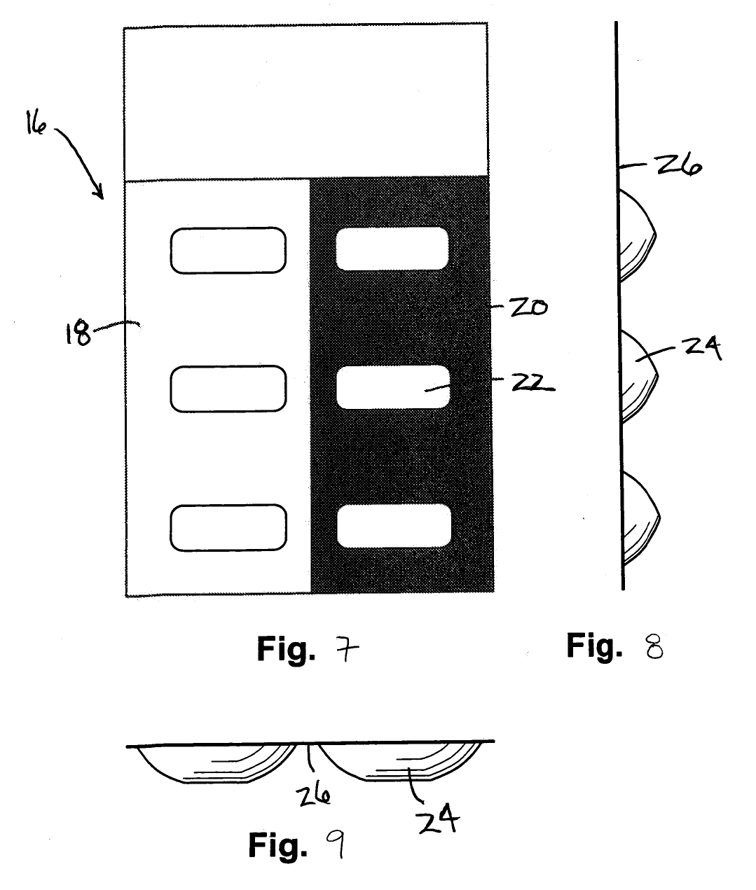

FIGS. 4-6 illustrate another embodiment of a color coded compliance aid of the present invention having two alternative shadings. FIG. 4 is a top plan view thereof showing alternating medium 8 and light 6 color shading regions and six windows 10 through which a capsule or tablet can be pushed to retrieve the capsule or tablet. FIG. 5 is a right side view thereof showing cavities 12 and bottom side 14. Cavities 12 would contain capsules or tablets. FIG. 6 is a front view thereof showing cavities 12 and bottom side 14.

FIGS. 7-9, FIGS. 10-12, and FIGS. 13-15 illustrate an embodiment of a color coded compliance aid having three shades of color that can be used to identify, for example, three different times of day. The color coded compliance aid comprises multiple blister packs. FIG. 7 is a top view of a blister pack 16 having a light 18 and a medium 20 shaded region, and having six windows 22 through which a capsule or tablet can be pushed to retrieve the capsule or tablet. FIG. 8 is a right side view thereof, showing cavities 24 on the bottom side 26. Cavities 24 would contain capsules or tablets. FIG. 9 is a front view thereof showing cavities 24 and bottom side 26.

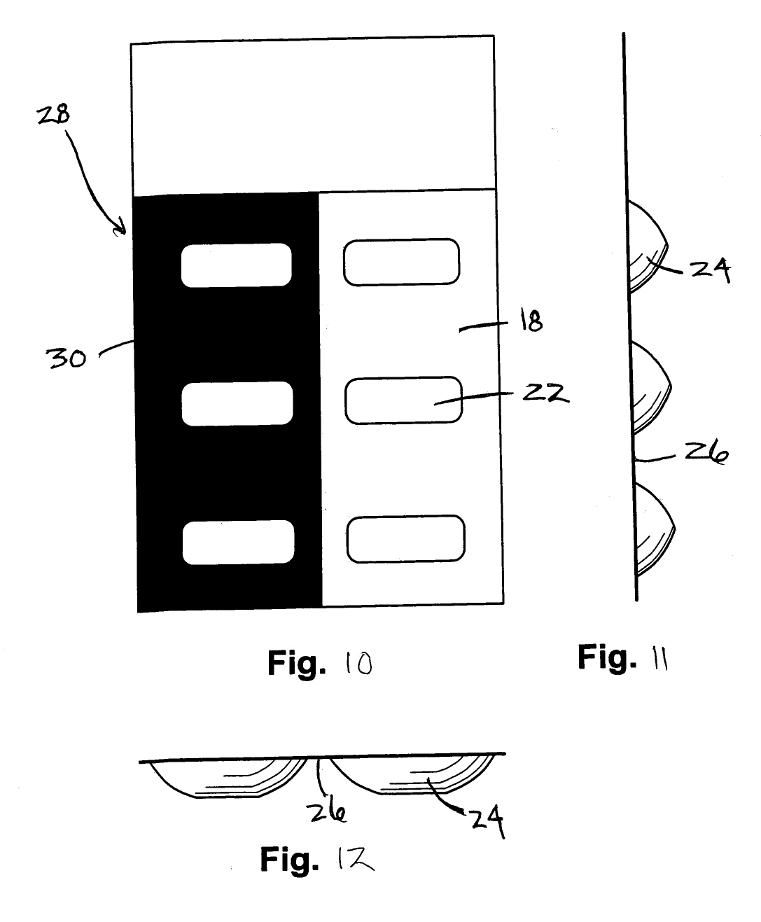

FIG. 10 is a top view of a blister pack 28 having a dark 30 and a light 18 shaded region, and having six windows 22 through which a capsule or tablet can be pushed to retrieve the capsule or tablet. FIG. 11 is a right side view thereof, showing cavities 24 on the bottom side 26. Cavities 24 would contain capsules or tablets. FIG. 12 is a front view thereof showing cavities 24 and bottom side 26.

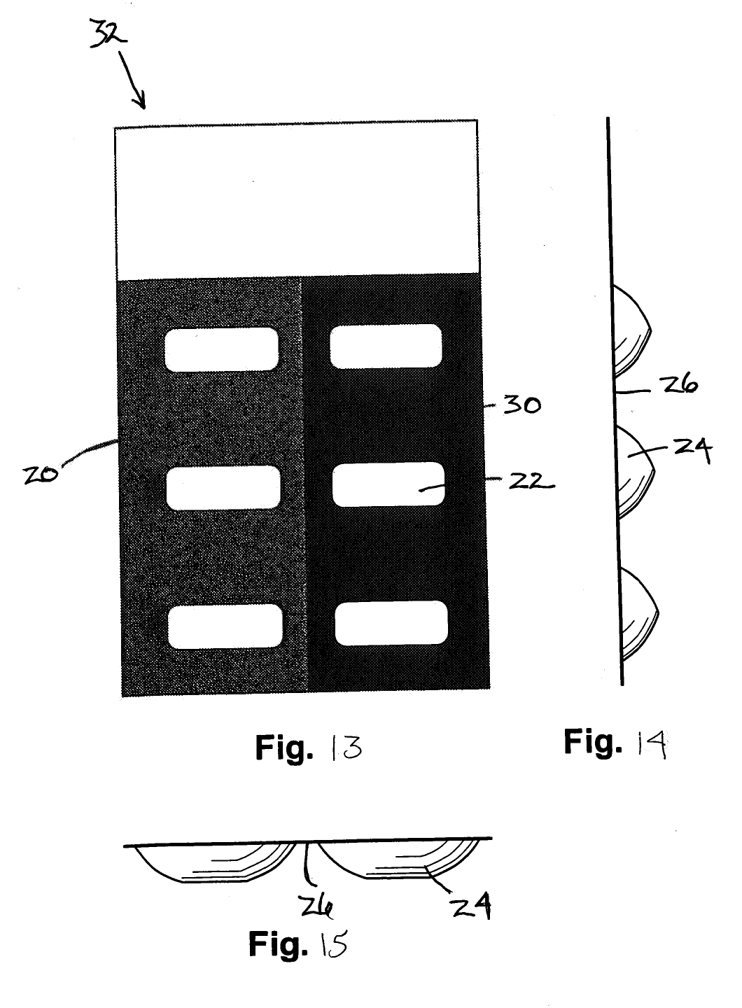

FIG. 13 is a top view of a blister pack 32 having a medium 20 and a dark 30 shaded region, and having six windows 22 through which a capsule or tablet can be pushed to retrieve the capsule or tablet. FIG. 14 is a right side view thereof, showing cavities 24 on the bottom side 26. Cavities 24 would contain capsules or tablets. FIG. 15 is a front view thereof showing cavities 24 on bottom side 26.

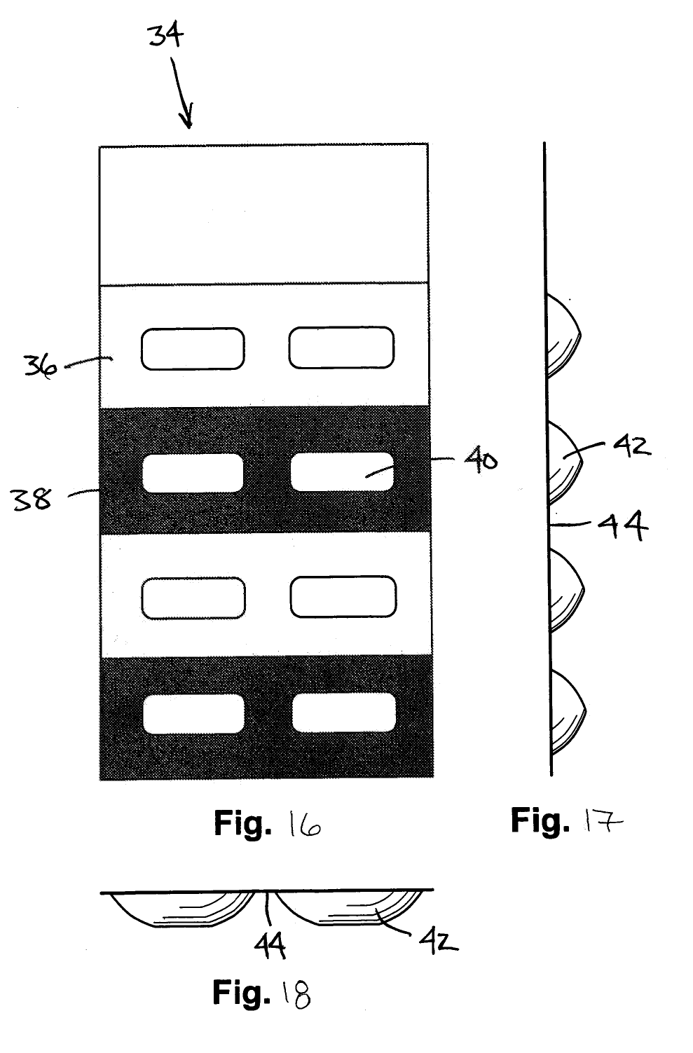

FIGS. 16-18 illustrate a different embodiment of a color coded compliance aid having two shades of color. FIG. 16 is a top view of a blister pack 34 having a plurality of alternating light 36 and medium 38 shaded regions, and eight windows 40 through which a capsule or tablet can be pushed to retrieve the capsule or tablet from the blister pack. FIG. 17 is a right side view thereof, showing cavities 42 on the bottom side 44. Cavities 42 would contain capsules or tablets. FIG. 18 is a front view thereof showing cavities 42 on bottom side 44.

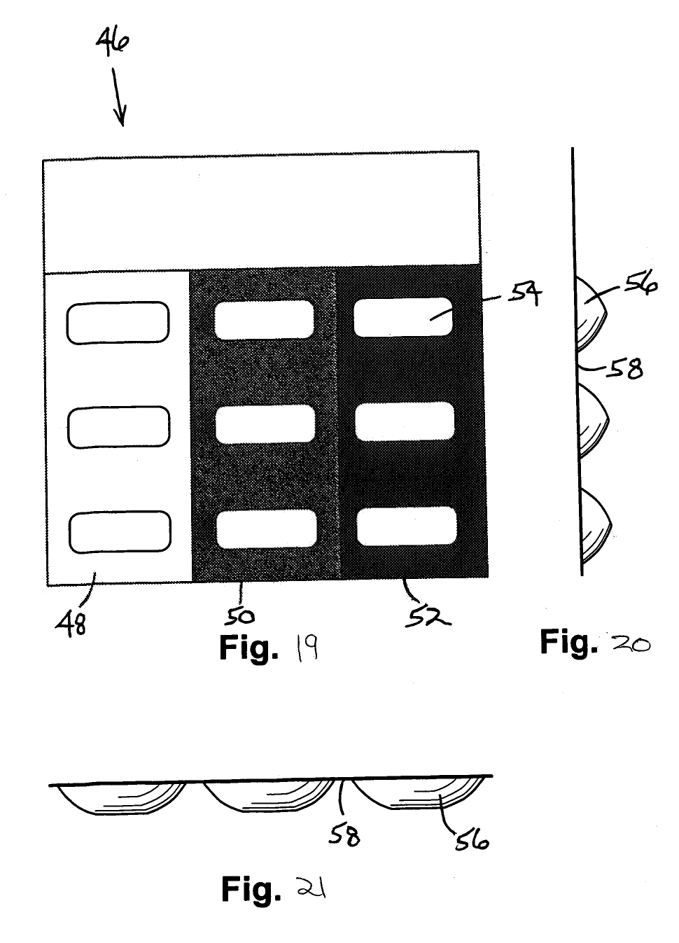

FIGS. 19-21 illustrate another embodiment of a color coded compliance aid having three shades of color. However, in this embodiment, three shades of color are used on a single blister pack. FIG. 19 is a top view of a blister pack 46 having a light 48, medium 50 and dark 52 shaded region thereon. Also shown are nine windows 54 through which a capsule or tablet can be pushed to retrieve the capsule or tablet from the blister pack. FIG. 20 is a right side view thereof, showing cavities 56 on the bottom side 58. Cavities 56 would contain capsules or tablets. FIG. 21 is a front view thereof showing cavities 56 on bottom side 58.

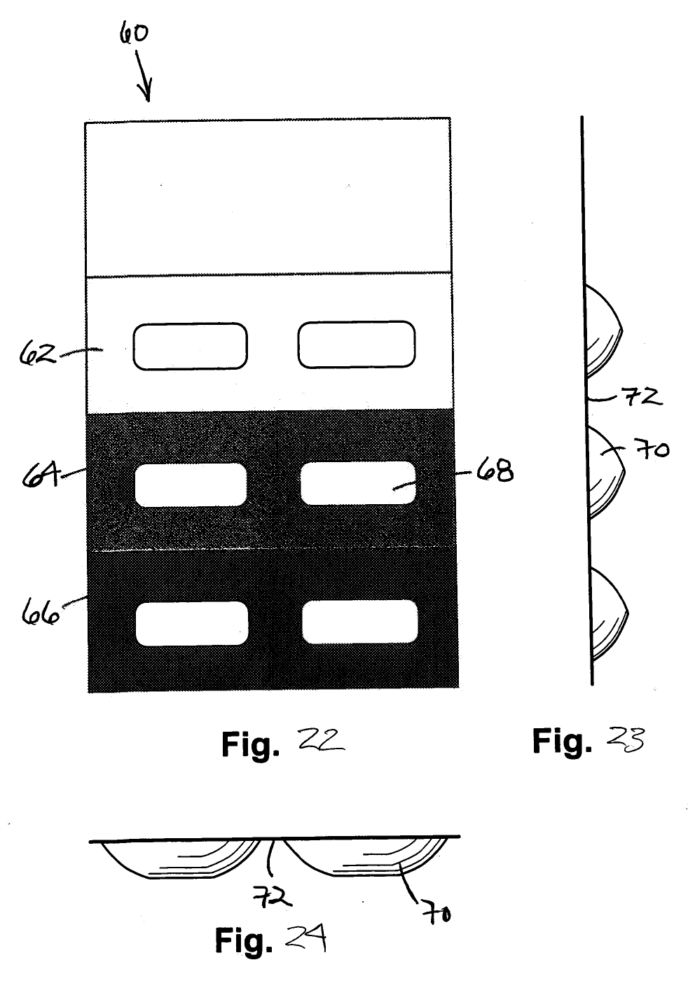

FIGS. 22-24 illustrate a different embodiment of a color coded compliance aid having three shades of color. FIG. 22 is a top view of a blister pack 60 having a light 62, medium 64, and dark 66 shaded region thereon. Also shown are six windows 68 through which a capsule or tablet can be pushed to retrieve the capsule or tablet from the blister pack. FIG. 23 is a right side view thereof, showing cavities 70 on the bottom side 72. Cavities 70 would contain capsules or tablets. FIG. 24 is a front view thereof showing cavities 70 on bottom side 72.

### EXAMPLES

The following non-limiting examples illustrate the methods and kits of the present invention.

### Example 1

A female with recurring digestive upsets and a history of untoward effects with available products is treated by method of the invention, and is referred to as “the user” of the method. The method includes a kit that contains a two month loading program with a set of labeled preparations and instructions for each week of the program. During Week One, four times daily between meals, the user prepares an infusion of chamomile botanical by pouring one sachet labeled “Week One” into a cup, pouring boiling water over the botanical, waiting 10 minutes, straining and then ingesting the infusion. During Week Two, the user continues with the chamomile infusion four times daily between meals, using “Week Two” labeled chamomile sachets, while additionally mixing one “Week Two” labeled prebiotic sachet containing inulin (5 grams per sachet) into a food or beverage of choice and fully consuming the prebiotic preparation three times daily. During Week Three, the user continues with the prebiotic three times daily with a food or beverage using the “Week Three” labeled prebiotic sachets while additionally beginning a loading dose of loading probiotic capsules each containing 1×10⁹ cfu Bifidobacterium infantis NCIMB 41003 in divided doses of 3 capsules per each dose, for a total of 9 capsules each day as provided on a “Week Three” color coded blister pack. Based on the level of digestive discomfort, symptoms, and/or negative adjustment effects the user might continue to experience (or because the user is not at her desired “target level” of relief of symptoms as indicated on the tracking/compliance device provided with the kit), the user continues to prepare and ingest an infusion of chamomile daily by using the enclosed “As Needed” labeled chamomile sachets. During Week Four, the user continues with the prebiotic three times daily with food or beverage using the “Week Four” labeled prebiotic sachets, and continues with a modified, tapered loading dose of loading probiotic capsules each containing 1×10⁹ cfu Bifidobacterium infantis NCIMB 41003 in divided doses of 2 capsules per each dose, for a total of 6 capsules each day as provided on a “Week Four” labeled color coded blister pack. Based on the level of digestive discomfort, symptoms, and/or negative adjustment effects the user might continue to experience (or because the user is not at her desired “target level” of relief of symptoms as indicated on the compliance aid provided with the kit), the user continues to prepare and ingest an infusion of chamomile daily by using the enclosed “As Needed” labeled chamomile sachets. During Weeks Four to Eight, the user takes a modified, tapered loading dose of one loading probiotic capsule containing 1×10⁹ cfu Bifidobacterium infantis NCIMB 41003 each day as provided on a color coded blister pack, each pack containing seven capsules. The user continues to use chamomile infusions as needed based on the perceived level of digestive discomfort or progress towards the desired level of improvement as indicated by daily monitoring using a provided compliance aid. After Week Eight, for a potentially indefinite maintenance time period, the user obtains and continues taking a dose of one maintenance probiotic capsule containing 1×10⁹ cfu Bifidobacterium infantis NCIMB 41003 each day as provided on a color coded blister pack, each pack containing seven capsules. The user continues to use chamomile infusions as needed based on the perceived level of digestive discomfort or the desired maintenance level of wellbeing as indicated by daily monitoring using a provided compliance aid.

### Example 2

An adult male with frequent complaints of excess gas and bloating is treated by a method of the invention, and is referred to as “the user” of the method. Each day for 28 days of a loading time period, the user ingests orally a loading dose of 9 capsules each containing 1×10⁹ cfu of Bifidobacterium infantis strain NCIMB 41003, and also ingests 1 capsule containing 550 mg of ground Ginger root each day. Immediately following this 28 day regimen, the user orally ingests a maintenance dose of 1 capsule containing 1×10⁹ cfu of Bifidobacterium infantis strain NCIMB 41003 and 1 capsule containing 550 mg of Ginger root, The user ingests the probiotic and/or the ginger indefinitely, daily, or as needed, for a maintenance time period.

### Example 3

A female with irritable bowel syndrome (IBS) of alternating bowel type (diarrhea and constipation) accompanied by bloating, abdominal cramping and diminished energy is treated by a method of the invention, and is referred to as “the user” of the method. To help initially calm her cramping, for a pre-loading time period a pre-loading regimen of 30 drops three times a day of a tincture of equal parts of the botanicals slippery elm, licorice, fennel seed, ginseng and valerian is initiated for at least 2 days and continued until the IBS-accompanying symptoms are sufficiently alleviated as perceived by the user. Next, the user begins a loading dose regimen of capsules of loading probiotic, for a loading time period, comprised of a daily dosage of 3 capsules each containing 1×10¹⁰ cfu Bifidobacterium infantis NCIMB 41003 and 3 capsules each containing 1×10¹⁰ cfu Lactobacillus plantarum 299v for one week, followed by 2 capsules of each loading probiotic daily for one week, followed by one capsule of each loading probiotic daily for one week or until bowel habits are satisfactorily normalized, as perceived by the user and/or tracked on a compliance aid. Following the loading time period, the user ingests a maintenance dose of one capsule containing 1×10¹⁰ cfu Bifdobacterium infantis NCIMB 41003 daily for an indefinite maintenance time period. The botanical tincture can be used on an as-needed basis throughout the loading and/or maintenance time periods to prevent or alleviate additional gastrointestinal symptoms that may arise, perhaps related to IBS or other stressors.

### Example 4

An adult female who is beginning work at a daycare center is concerned with her exposure to upper respiratory infections, in particular the common cold, desires to boost her immune system and is treated by a method of the invention. She is referred to as “the user” of the method. Each day for 14 days of a loading time period, the user ingests orally a loading dose of 3 capsules each containing 1×10⁹ cfu of Lactobacillus rhamnosusGG, and concomitantly ingests 3 times each day 3 capsules containing 450 mg of licorice root. Immediately following this 14 day regimen, each day the user orally ingests a maintenance dose of 1 capsule containing 1×10⁹ cfu of Lactobacillus indefinitely for a maintenance time period.

### Example 5

An adult female with fibromyalgia is treated by a method of the invention, and is referred to as the “user” of the method. Each day for 21 days of a loading time period, the user ingests orally a loading dose of 1 capsule containing 1×10¹² cfu of Bifidobacterium infantis strain NCIMB 41003, and concomitantly orally dissolves a lozenge containing 150 mg of slippery elm bark coated with a cooling sensate every two hours as needed for heartburn and/or negative adjustment effects related to the loading dose of the loading probiotic. Immediately following this 21 day regimen, the user orally ingests, daily, a maintenance dose of 1 capsule containing 1×10⁹ cfu of Bifidobacterium infantis strain NCIMB 41003 and continues use of the lozenge if desired, ingesting both for a maintenance time period.

### Example 6

An adult female who has been suffering from gastrointestinal upsets (episodes of diarrhea followed by constipation with concomitant gas and bloating), feels extremely drained and tired all of the time. She attributes her tiredness to her digestive upsets. She is treated by a method of the invention and is referred to as the “user” of the method. Each day for 21 days of a loading time period, the user ingests orally a loading dose of 2 capsules each containing 5×10⁹ cfu of Bifidobacterium infantis strain NCIMB 41003, and concomitantly swallows a tablet containing 500 mg Vitamin C, 30 I.U. Vitamin E, 10 mg Thiamin, 10 mg Riboflavin, 100 mg Niacin, 5 mg B6, 400 mcg Folic Acid, 12 mcg Vitamin B12, 45 mcg Biotin, 20 mg Pantothenic Acid, 23.9 mg Zinc, and 3 mg Copper. Immediately following this 21 day regimen, the user orally ingests, daily, a maintenance dose of 1 capsule containing 1×10⁹ cfu of Bifidobacterium infantis strain NCIMB 41003 for a maintenance time period.

### Example 7

An adult male who has been suffering from diarrhea feels depressed, tired, and mentally drained with impaired mentation. He attributes his mentation problems to years of suffering from malnourishment as a result of the diarrhea. He is treated by a method of the invention and is referred to as the “user” of the method. Each day for 14 days of a loading time period, the user ingests orally a loading dose of 1 capsule containing 1×10¹⁰ cfu of Bifidobacterium infantis strain NCIMB 41003, and concomitantly swallows a tablet of Stress B-Complex available from Nature Made Nutritional Products, Mission Hills, Calif., USA and containing 2.0 mg Thiamin, 2.1 mg Riboflavin, 40 mg of Niacin, 4 mg of B6, 550 mcg Folic Acid, 10 mcg Vitamin B12, 60 mcg Biotin, 12 mg of Zinc, 4000 IU Vitamin A, 400 IU Vitamin D, 50 mcg Vitamin K, 100 mg Vitamin C, 250 mg Calcium, 15 mg Iron, and 0.9 mg Copper. As needed, he also drinks an infusion of rosemary, ginger and ginseng. Immediately following this 14 day regimen, the user orally ingests, daily, a maintenance dose of 1 capsule containing 1×10⁹ cfu of Bifidobacterium infantis strain NCIMB 41003.

### Example 8

A dog with frequent loose stools is treated by a method of the invention, and is referred to as “the user” of the method. Each day for 28 days of a loading time period, the user is administered and orally ingests a loading dose of 5 capsules each containing 1×10⁹ cfu of Bifidobacterium pseudolongum strain NCIMB 41199, and also ingests 1 capsule containing 2 mg of β-carotene each day. Immediately following this 28 day regimen, the user is administered and orally ingests a maintenance dose of 1 capsule containing 1×10⁹ cfu of Bifidobacterium pseudolongum strain NCIMB 41199 and 1 capsule containing 2 mg of β-carotene. The user ingests the probiotic and/or the β-carotene indefinitely, daily, or as needed, for a maintenance time period.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mg” is intended to mean “about 40 mg”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of administering a probiotic comprising the steps of:
a. administering a loading dose of a loading probiotic for a loading time period; and
b. administering a dose of a botanical for said loading time period.

2. The method of claim 1 further comprising administering an additional material.

3. The method of claim 1 wherein said loading probiotic comprises lactic acid bacteria selected from the group consisting of Bifidobacterium, Lactobacillus, Streptococcus and combinations thereof.

4. The method of claim 3 wherein said lactic acid bacteria comprises an isolated strain of Bifidobacterium infantis.

5. The method of claim 1 wherein said loading probiotic is administered to provide from about 1×10³ to about 1×10¹⁴ cfu of loading probiotic per day.

6. The method of claim 1 wherein said loading probiotic is administered to provide from about 1×10⁵ to about 1×10¹⁴ cfu of loading probiotic per day.

7. The method of claim 1 wherein said loading probiotic is administered to provide from about 1×10⁹ to about 1×10¹² cfu of loading probiotic per day.

8. The method of claim 1 wherein said probiotic is administered in a dosage form selected from the group consisting of: capsule, chewable tablet, swallowable tablet, buccal tablet, troche, powder, lozenge, soft chew, solution, suspension, spray, tincture, decoction, infusion, syrup, elixir, wafer, food product, and combinations thereof.

9. The method of claim 8 wherein said food product is selected from the group consisting of acidified milk, yogurt, milk powder, tea, juice, beverage, confection, chewable bar, cookie, wafer, cracker, cereal, soft chew, and combinations thereof.

10. The method of claim 1 wherein said botanical exerts benefits on the gastrointestinal system, selected from the group consisting of: soothing effects, demulcent effects, gas reducing effects, carminative effects, anti-diarrheal effects, astringent effects, laxative effects, aperient effects, cathartic effects, purgative effects, hydrogogue effects, analgesic effects, antispasmodic effects, relaxation effects, stimulant effects, bitter effects, digestive aid effects, health effects, and combinations thereof.

11. The method of claim 1 wherein said botanical is selected from the ginger Family, Licorice root, Marshmallow root, Chamomile, Fennel oil, Fennel Seed, Caraway oil, Caraway seed, Lemon Balm, Horehound Herb, Flaxseed, Flaxseed alpha-linoleic acid, Rosemary Leaf, Rosemary extract, Polyphenols, Avocado extract, Mannoheptulose, and combinations thereof.

12. The method of claim 1 wherein said botanical is provided in a form selected from the group consisting of an extract, tincture, oil, fresh root or rhizome, dried root or rhizome, powdered root or rhizome, whole root or rhizome, infusion, decoction, crystallized matter and combinations thereof.

13. The method of claim 1 wherein said dose of said botanical for said loading time period comprises from about 0.001 g to about 100 grams of said botanical per day.

14. The method of claim 1 wherein said botanical is administered in a dosage form selected from the group consisting of: capsule, chewable tablet, swallowable tablet, coated tablet, buccal tablet, powder, lozenge, soft chew, solution, suspension, spray, extract, tincture, oil, decoction, infusion, syrup, elixir, food product, and combinations thereof.

15. The method of claim 14 wherein said food product is selected from the group consisting of: acidified milk, yogurt, milk powder, tea, juice, beverage, confection, chewable bar, cookie, wafer, cracker, cereal, soft chew, treat, and combinations thereof.

16. The method of claim 1 wherein said loading probiotic and said botanical are administered together in a dosage form.

17. The method of claim 2 wherein said additional material is selected from the group consisting of vitamins, minerals, metals, elements, essential fatty acids, essential amino acids, sensates, prebiotics, carotenoids, and combinations thereof.

18. The method of claim 2 wherein said additional material is administered in an amount of from about 0.001 μg to about 10 g of said additional material, per day.

19. The method of claim 1 further comprising using a compliance aid to track, assess and improve a user's use of and compliance with said method.

20. The method of claim 1 wherein said loading time period comprises from about 1 day to about 60 days.

21. The method of claim 1 further comprising administering a maintenance dose of a maintenance probiotic for a maintenance time period.

22. The method of claim 21 wherein said maintenance dose of said maintenance probiotic during said maintenance time period is an amount effective to maintain alleviation of symptoms.

23. The method of claim 21 wherein said maintenance probiotic comprises lactic acid bacteria selected from the group consisting of Bifidobacterium, Lactobacillus, Streptococcus and combinations thereof.

24. The method of claim 23 wherein said lactic acid bacteria comprises an isolated strain of Bifidobacterium infantis.

25. The method of claim 21 wherein said maintenance probiotic is administered for said maintenance time period to provide from about 1×10³ to about 1×10¹² cfu of maintenance probiotic per day.

26. The method of claim 21 further comprising administering a dose of said botanical for said maintenance time period.

27. The method of claim 26 wherein said dose of said botanical for said maintenance time period comprises from about 0.001 g to about 100 g of said botanical per day.

28. The method of claim 21 further comprising administering an additional material or said maintenance time period.

29. The method of claim 28 wherein said additional material is administered in an amount of from about 0.001 μg to about 10 g of said additional material, per day.

30. The method of claim 1 further comprising administering a pre-loading composition for a pre-loading time period before said loading time period.

31. The method of claim 30 wherein said pre-loading composition is selected from the group consisting of: a probiotic, a botanical, an additional material, and combinations thereof.

32. A method of administering a probiotic comprising the steps of:
a. administering a loading dose of a loading probiotic for a loading time period; and
b. administering a dose of an additional material for said loading time period.

33. The method of claim 32 wherein said additional material is selected from the group consisting of: vitamins, minerals, metals, elements, essential fatty acids, essential amino acids, sensates, prebiotics, and combinations thereof.

34. A method of administering a probiotic comprising the steps of:
a. administering a loading dose of a loading probiotic for a loading time period;
b. administering a dose of a botanical for said loading time period; and
c. administering a dose of an additional material for said loading time period.

35. A method of administering a probiotic comprising the steps of:
a. administering a loading dose of a loading probiotic for a loading time period;
b. administering a dose of a botanical for said loading time period;
c. subsequently administering a dose of a maintenance probiotic for a maintenance time period.

36. The method of claim 35 further comprising administering a dose of a botanical for said maintenance time period.

37. The method of claim 35 further comprising administering a dose of an additional material for said loading time period.

38. The method of claim 35 further comprising administering a dose of an additional material for said maintenance time period.

39. The method of claim 35 further comprising, prior to steps a and b, administering a pre-loading composition for a pre-loading time period.

40. A method of administering a probiotic comprising the steps of:
a. administering a loading dose of a loading probiotic for a loading time period;
b. administering a dose of an additional material for said loading time period;
c. subsequently administering a dose of a maintenance probiotic for a maintenance time period.

41. The method of claim 40 further comprising administering a dose of an additional material for said maintenance time period.

42. The method of claim 40 further comprising administering a dose of a botanical for said loading time period.

43. The method of claim 40 further comprising administering a dose of a botanical for said maintenance time period.

44. The method of claim 40 further comprising, prior to steps a and b, administering a pre-loading composition for a pre-loading time period.

45. A kit for use in administering a probiotic comprising:
a. loading doses of a loading probiotic to be administered for a loading time period; and
b. doses of a botanical to be administered for said loading time period.

46. The kit of claim 45 further comprising instructions for use of said kit.

47. The kit of claim 45 further comprising a compliance aid.

48. The kit of claim 45 further comprising doses of an additional material to be administered for said loading time period.

49. The kit of claim 45 further comprising doses of a maintenance probiotic to be administered for a maintenance time period.

50. The kit of claim 49 further comprising doses of a botanical to be administered for said maintenance period time period.

51. The kit of claim 49 further comprising doses of an additional material to be administered for said maintenance time period.

52. The kit of claim 45 further comprising doses of a pre-loading composition to be administered for a pre-loading time period.

53. A kit for use in administering a probiotic comprising:
a. loading doses of a loading probiotic to be administered for a loading time period; and
b. doses of an additional material to be administered for said loading time period.

54. A kit for use in administering a probiotic comprising:
a. loading doses of a loading probiotic to be administered for a loading time period;
b. doses of a botanical to be administered for said loading time period; and
c. doses of an additional material to be administered for said loading time period.
